(19) United States
(12) Patent Application Publication
(10) Pub. No.: US 2013/0059354 A1
(43) Pub. Date: Mar. 7, 2013

(54) ALKANOL

(71) Applicants: Børre Tore Børresen, Heimdal (NO); Hans Kristian Kotlar, Heimdal (NO)

(72) Inventors: Børre Tore Børresen, Heimdal (NO); Hans Kristian Kotlar, Heimdal (NO)

(73) Assignee: STATOIL PETROLEUM AS, Stavanger (NO)

(21) Appl. No.: 13/575,618

(22) PCT Filed: Feb. 14, 2011
(86) PCT No.: PCT/EP11/52146

§ 371 Date: Sep. 27, 2012

(30) Foreign Application Priority Data
Feb. 16, 2010 (GB) 1002609.4

(51) Int. Cl. C12P 7/04; C12P 7/16; C12M 1/00

(52) U.S. Cl. 435/160; 435/157; 435/294.1

(57) ABSTRACT

The present invention provides a process for the production of an alkanol (e.g. butanol) comprising converting a carbon source such as a biomass into a carboxylic acid or a derivative thereof using a microorganism, and electrochemically reducing said acid to said alkanol, as well as an apparatus for carrying out said process.

The present invention relates to processes for the production of alkanol, and in particular, butanol from a carbon source such as biomass. The invention further relates to an apparatus for carrying out the processes of the present invention.

### BACKGROUND

Alkanols, and more precisely, C₁₋₆ monohydric alkanols, especially ethanol and butanol are of increasing importance as fuels, either per se or as additives to conventional hydrocarbon fuels such as gasoline. The production of ethanol for use as a fuel in this way has recently received much attention. However butanol, and especially 1-butanol, 2-butanol and isobutanol, is more attractive as a fuel option than ethanol. Butanol is more readily mixed with conventional liquid hydrocarbon fuel than ethanol and its calorific yield on combustion is higher than that of ethanol.

Yeasts, e.g. Brewer's yeast, has long been known to be capable of converting fermentable sugars to alcohols such as ethanol and bacteria such as Clostridium have long been known to be capable of converting fermentable sugars to alcohols such as ethanol, propanol and butanol. The alcohols produced by these fermentations may be separated off from the fermentation mixture by, for example, distillation.

Unfortunately, however, the separation process to isolate alcohols from such fermentation reactions requires considerable amounts of energy. This is because the yeast and bacteria used to convert sugars into alkanols are poisoned by the alkanols, and other bi products, at relatively low concentrations of alkanol and cease alkanol production. This means that the maximum concentration of ethanol and butanol that can be achieved using conventional yeast and bacteria is about 13% and 2% respectively. Consequently a significant amount of energy is required to remove relatively low percentages of alkanol from water.

More recently considerable attention has been paid to the possibility of fermenting sugars derived from biomass into alkanols. Typically biomass is treated with an acid to produce fermentable sugars that can subsequently be converted into alkanols. Many different organisms, including genetically modified organisms, have been developed to try to increase the amount of alkanol that can be obtained from such processes. The isolation of alkanol from the fermentation broth still, however, requires significant amounts of energy making such processes energy costly.

To try to address this problem, instead of producing alkanol directly from biomass, it has been proposed to convert biomass or fermentable carbohydrates to acid, such as ethanoic acid and butanoic acid by fermentation and then to convert the acid by catalytic hydrogenation or hydrogenolysis into alkanol. US2006/0019360, for instance, discloses such a process for the production of ethanol and US2008/0248540 discloses an analogous process for the production of butanol.

More specifically US2008/0248540 discloses a process for the production of butanol wherein a biomass feedstock is first converted to butyric acid using butyric acid producing bacteria such as Clostridium tyrobutyricum at about 37° C. The butyric acid present in the fermentation broth is recovered and purified by an extraction with an aliphatic amine followed by stripping of the solvent with hot water or steam. Thus partially purified butyric acid is obtained for use in a catalytic hydrogenation.

The hydrogenation of butyric acid to butanol, however, requires the use of elevated temperatures and pressures to achieve acceptable productivity and/or the use of expensive catalysts. US2008/0248540, for example, teaches the use of selective, supported catalysts, e.g. a bimetallic catalyst consisting of a Group VIII late transition-metal compound, a Ru—Sn system and a MgO—NH₂—Ru system. Nevertheless the hydrogenation reaction still requires elevated temperatures (150-200° C.) and pressures (200-300 atmospheres) as well as an input of hydrogen. US2008/0248540 describes the possibility of using the hydrogen produced in fermentation in the hydrogenation process but this is challenging to achieve in practice. At least some additional hydrogen is likely to be required and the production of hydrogen is costly. Moreover the butanol that results from the process described in US2008/0248540 still has to be recovered via an energy consuming distillation process.

### SUMMARY OF INVENTION

Thus viewed from a first aspect the present invention provides a process for the production of an alkanol comprising:
(i) converting a carbon source into a carboxylic acid or a derivative thereof using a microorganism; and
(ii) electrochemically reducing said acid to said alkanol.

In a preferred embodiment of the invention, the acid is butanoic acid, preferably 1-butanoic acid, 2-butanoic acid or isobutanoic acid and the alkanol is butanol, preferably 1-butanol, 2-butanol or isobutanol.

Viewed from a further aspect the present invention provides an apparatus for producing an alkanol comprising:
(ia) a first reactor for converting a carbon source into a carboxylic acid or a derivative thereof using a microorganism; and
(ib) a second reactor for electrochemically reducing said acid to said alkanol, wherein said first reactor is fluidly connected to said second reactor.

The processes and apparatus of the present invention are advantageous because:
1) the microorganisms are not exposed to high levels of alkanol, therefore poisoning does not occur
2) the electrochemical reduction is favoured by a high concentrations of acid and can produce higher concentrations of alcohol than microorganisms
3) the electrochemical reduction can optimally produce enough alkanol such that a two phase system is formed—one mainly water, one approx 1:1 alcohol:water—thereby facilitating recovery of the alkanol, i.e. reducing energy costs for distillation.

### DESCRIPTION

The process of the present invention is particularly useful for the production of alkanols of the formula ROH, wherein R is straight chain or branched alkyl. Particularly preferably R is C₁₋₈ alkyl, more preferably C₂₋₆ alkyl, in particular C₂ or C₄ alkyl, especially C₄ alkyl. The processes of the present invention are ideally suited for the preparation of ethanol (CH₃CH₂OH) and butanol, especially butanol. 1-Butanol (CH₃CH₂CH₂CH₂OH), 2-butanol (CH₃CH₂CHOHCH₃) and isobutanol ((CH₃)₂CHCH₂OH) production is particularly preferred.

In a preferred process of the present invention the carbon source is biomass. As used herein the term “biomass” refers to any renewable, organic matter collected for use as a source of energy. Representative examples of types of biomass include algal biomass, plant biomass, animal biomass (e.g. any animal by-product, animal waste, etc.) and municipal waste biomass (e.g. residential and light commercial refuse with recyclables such as metal and glass removed). Algal biomass and plant biomass, especially algal biomass, is generally preferred.

The term “algal biomass” as used herein is intended to refer to any algal-derived organic matter. It includes seaweed as well as microalgae (i.e. organisms capable of photosynthesis that are less than 1 mm in diameter). Algal biomass includes seaweeds such as brown seaweed (Phaeophyceae), red seaweed (Rhodophyceae), green seaweed (Chlorophyceae) and plantae such as Bacillariophyceae, the green algae (Chlorophyceae), the blue-green algae (Cyanophyceae) and the golden algae (Chrysophyceae). Seaweed is a particularly preferred type of biomass for use in the processes of the present invention. Seaweed generally comprises the carbohydrate laminaran which comprises gluclose and mannitol sugars linked by β1-3 and β1-6 glycosidic bonds and alginate which comprises mannuronate and guluronate sugars linked by β1-4 bonds.

The term “plant biomass” as used herein is intended to refer to any plant-derived organic matter (woody or non-woody) available for energy on a sustainable basis. Plant biomass includes agricultural crop wastes and residues such as corn stover, casava, corn cob, wheat straw, wheat bran, rice straw, sugar cane bagasse etc. Plant biomass also includes woody energy crops, wood wastes and residues such as trees, softwood forest thinnings, barky wastes, sawdust, paper and pulp industry waste streams, wood fiber, etc. Additionally grass crops, such as switch grass, have potential to be produced large scale as another plant biomass source. For urban areas, the best potential plant biomass feedstock comprises garden waste (e.g., grass clippings, leaves, tree clippings, brush, etc.) and vegetable processing waste. Plant biomass is known to be the most prevalent form of carbohydrate available in nature.

Plant biomass generally comprises three primary chemical fractions: hemicellulose, cellulose and lignin. Hemicellulose is a polymer of short, highly-branched chains of mostly five-carbon pentose sugars (e.g. xylose and arabinose) and to a lesser extent six-carbon hexose sugars (e.g. galactose, glucose and mannose). These sugars are highly substituted with acetic acid. Hemicellulose has a highly branched structure and is therefore amorphous and relatively easy to hydrolyze (or cleave) to its individual constituent sugars, e.g. by enzyme or dilute acid treatment. Cellulose is a linear polymer of glucose sugars connected by beta-glycosidic linkages. This allows cellulose to form closely-associated hydrogen bonded linear chains. Cellulose therefore forms a rigid crystalline structure that is highly stable and much more resistant to hydrolysis by chemical or enzymatic attack than hemicellulose polymers. Lignin, which is a polymer of phenolic molecules, provides structural integrity to plants, and typically remains as residual material after the sugars in plant biomass have been fermented.

Cellulose makes up a significant proportion, e.g. 30 to 50% of most kinds of biomass. Thus whilst cellulose is more difficult to convert to sugars than hemicellulose, its efficient use is essential for maximizing alkanol yields, such as butanol, on a per ton basis of plant biomass. Preferred plant biomass for use in the process of the present invention comprises at least 50% by weight, more preferably at least 60% by weight, still more preferably at least 70% by weight cellulose and hemicellulose. The maximum % weight of hemicellulose and cellulose might be 95% or 99%.

Preferred methods of the invention comprise a pre treatment step wherein biomass is converted to sugars, e.g. fermentable sugars. Thus in a preferred process of the invention step (i) comprises the steps of:
(ia) converting a carbon source into sugars; and
(ib) treating said sugars with the microorganism to yield said carboxylic acid or a derivative thereof.

As used herein the term “sugar”, and more particularly “fermentable sugar”, is intended to encompass any sugar capable of being converted by a microorganism into an alkanol. Preferred sugars are pentoses and hexoses, e.g. xylose, arabinose, galactose, glucose, mannose, mannitol, mannuronate and guluronate.

Any conventional pre treatment step may be used to convert carbon source to sugars. Pre treatment methods can, for example, utilise acids of varying concentrations (including sulfuric acids, hydrochloric acids, organic acids, etc.) and/or other chemicals such as ammonia, ammonium and lime. Pre treatment methods can additionally or alternatively employ hydrothermal treatments including water, heat, steam or pressurised steam and/or various enzymes, e.g. enzymatic hydrolysis. Thermally stable enzymes may, for example, be used to convert carbon source to sugars.

Preferred pre treatment steps for use in the present invention include an acid (e.g. dilute or concentrated) hydrolysis treatment, hydrothermal treatment (e.g. steam explosion and hot water extraction), chemical treatment (e.g. ammonia explosion, ammonia recycled percolation (ARP), lime treatment), enzymatic hydrolysis (e.g. using thermally stable enzymes) or any combination of the afore-going.

Such pre treatment steps may be carried out using conventional methods and equipment. Acid hydrolysis may, for example, be carried out by mixing biomass with acid (e.g. 0.1-1 M HCl or H₂SO₄). Typically the mixture is heated, e.g. heated to 75 to 125° C. and optionally pressurised. A typical reaction time might be 0.5-5 hours. After hydrolysis is complete, insoluble materials are removed by filtration and the hydrolysate (e.g. aqueous sugar solution) collected. Preferably the hydrolysate is neutralised before it is used in subsequent steps. The hydrolysate is preferably sterilised before use in subsequent steps.

Alternatively, or additionally, a hydrothermal treatment may be carried out by mixing a biomass with water and then heating the mixture by steam explosion. The mixture is preferably heated to temperatures in the range 80 to 150° C. Preferably the process is carried out under pressures of 5 to 30 bar. After treatment is complete, insoluble material is preferably removed by filtration. Sugars, e.g. an aqueous sugar solution, may be collected.

Another preferred pre treatment that may be used in the process of the invention is treating said carbon source with one or more microorganisms that convert carbon source to sugars. The microorganisms may be mesophilic or thermophilic. By “mesophilic” is meant herein that the microorganism is capable of proliferating in aqueous solution at a temperature between 15 to 45° C., more preferably 20 to 40° C., still more preferably 25 to 35° over a prolonged period, e.g. at least 10 hours. By “thermophilic” is meant herein that the microorganism is capable of proliferating in aqueous solution at a temperature of at least 45° C. over a prolonged period, e.g. at least 10 hours, preferably a temperature of at least 50° C., more preferably at least 60° C., especially 60 to 80° C. Typically the microorganism is used in the form of an aqueous inoculate. The skilled man can readily prepare suitable inoculates.

The microorganism used is preferably thermophilic. Thus step (i) preferably comprises treating a carbon source with a thermophilic microorganism at a temperature of at least 45° C., more preferably at least 50° C., still more preferably at least 60° C., especially 60 to 80° C., to yield said carboxylic acid or a derivative thereof. The use of thermophilic microorganisms advantageously eliminates the need for any neutralisation steps after conversion is complete.

The thermophilic microorganisms used for carbon source, e.g. cellulose, breakdown may be any thermophilic microorganism capable of achieving this conversion. Suitable microorganisms may be found in the hot centre of any compost heap. Highly thermophilic microorganisms may be isolated by cultivating a sample from such an environment at successively higher temperatures, e.g. raising the temperature in 5° C. increments from 35° C. to the desired operating temperatures. Alternatively, and generally more preferably, such organisms may be isolated from source materials by incubating at the desired, elevated operating temperatures. A preferred microorganism species is Clostridium. Examples of useful microorganism species include Clostridium thermocellum, C. thermosaccharolyticum, C. stercorarum, C. straminisolvens, and C. thermoamylolyticum, especially C. thermocellum DSM1237, C. stercorarum DSM8532, C. straminisolvens DSM16021, and C. thermoamylolyticum DSM2335 (see also Ozkan et al., J. Ind. Micribiol. & Biotech. 27:275-280 (2001)).

Suitable cellulose breakdown promoting thermophilic microorganisms also include those producing cellulases, hydrolases, lactases and/or peroxidises. Clostridium strains having cellulose degrading enzymes are known (see for example Sakka et al, Agricultural and Biological Chemistry 53:905-910 (1989), and Kato et al Int. J. Syst. Evol. Microbiol. 54:2043-2047 (2004)), and may conveniently be used in the processes of the present invention.

When a thermophilic microorganism is used to convert carbon source to sugars, the carbon source, e.g. biomass, may additionally be pretreated by one of the methods hereinbefore described, e.g. by hydrothermal treatment. If a pretreatment is employed, the resulting mixture is typically heated, e.g. to at least 45° C., then an inoculate of microorganism added. If no pretreatment step is used, the biomass is typically mixed with water, optionally macerated, and heated to a suitable temperature, e.g. at least 45° C. and an inoculate added. In both cases, the mixture is preferably maintained at the chosen temperature for at least 24 hours, more preferably at least 48 hours, e.g. at least 72 hours. Preferably the mixture is maintained under an inert atmosphere, e.g. a nitrogen atmosphere. After treatment is complete, sugars generally in the form of an aqueous solution are obtained. The insoluble materials may be removed by filtration, e.g. by using a conventional filter press.

In a preferred process of the invention the sugars, e.g. fermentable sugars, produced from biomass are converted into a carboxylic acid or a derivative thereof by treatment with a microorganism. Preferably this process yields a carboxylic acid of the formula R′COOH, wherein R′ is straight chain or branched, preferably straight chain, alkyl. Particularly preferably R′ is C₁₋₇ alkyl, more preferably C₂₋₅ alkyl, in particular C₁ or C₃ alkyl. The processes of the present invention are ideally suited for the preparation of ethanoic acid (CH₃COOH) and butanoic acid, especially butanoic acid (CH₃CH₂CH₂COOH).

The processes of the present invention may also yield carboxylic acid derivatives. As used herein, the term “derivative” encompasses salts and esters of carboxylic acids. When a derivative is produced it is preferably a salt. Representative examples of salts include sodium, potassium, lithium and ammonium salts. Examples of esters include methyl, ethyl and butyl ester.

The microorganism used to convert a carbon source (e.g. sugar) into a carboxylic acid or derivative thereof may be any known bacteria or fungi capable of carrying out this conversion. Preferably the microorganism is a bacterium, particularly an anaerobic bacterium. A mixture of microorganisms may optionally be employed. The microorganism may be mesophilic or thermophilic.

The microorganism may, for example, be a Clostridium, Actinetobacter, Arthrobacter, Bacillus, Bifidobacteria, Butyrovibrio, Escherichia, Enterococcus, Eubacterium, Flavobacterium, Fusobacterium, Megaspheara, Nocardia, Pseudobutyrovibrio, Rhizobium, Rhodococcus, Roseburia, Streptomyces, Ralstonia, Taleabrevis, Ureibacillus, Thermoanaerobacter, Thermoanaerobacterium, Lactobacillus, Geobacillus, Pichia, Saccharomyces, Zymomonas, Pseudomonas, Alcaligenes, Klebsiella, Paenibacillus, Corynebacterium, Brevibacterium, Candida or Hansenula species.

Particularly preferred microorganisms for use in some processes of the present invention include Actinetobacter sp., Arthrobacter sp., Bacillus coagulans, Bacillus sp., Bacillus thermoglucosidasius TN-T9, Bifidobacterium adolescentis, Butyrivibrio hungatei, Butyrivibrio sp., Clostridium acetobutylicum, Clostridium acetobutylicum strain SA-1, Clostridium aurantibutyricum, Clostridium beijerinckii, Clostridium butyicum, Clostridium celerecrescens, Clostridium kluyveri, Clostridium ljungdahlii, Clostridium pastuerianum, Clostridium proteoclasticum, Clostridium saccharobutylicum, Clostridium tetanomorphum, Clostridium thermobutyricum, Clostridium saccharolyticum, Clostridium thermocellum, Clostridium thermosaccarolyticum, Clostridium tyrobutyricum, Clostridium carboxidivorans, Eubacterium limosum, Eubacterium spp., Flavobacterium nucleatum, Flavobacterium sp., Fusobacterium prauznitzii, Fusobacterium spp., Geobacillus thermoglucosidasius, Geobacillus Lactobacillus fermentum, Lactobacillus spp., Megasphaera elsdenii, Nocardia sp., Pseudobutyrovibrio ruminis, Psuedobutyrovibrio xylanivorans, Taleabrevis butyricans, Thermoanaerobacter ethanolicus and Thermoanaerobacterium thermosaccharolyticum.

Still more preferably the microorganism is of the genus Clostridium. Preferred Clostridium species include C. acetobutylicum, C. aurantibutyricum, C. beijerinckii, C. butyicum, C. kluyveri, C. pastuerianum, C. saccharobutylicum, C. saccharolyticum, C. thermobutyricum, C. thermocellum, C. the rmosaccarolyticum, Clostridium carboxidivorans and C. tyrobutyricum.

In a preferred embodiment of the invention, the microorganism, e.g. mesophilic or thermophilic microorganism, used for carboxylic acid production is a genetically modified form of a microorganism capable of producing a carboxylic acid ester from a carboxylic acid precursor, the genetic modification being to knock out (i.e. disable) or delete a gene responsible for the acid bioprecursor to ester conversion. In the case of Clostridium for example this may involve knocking out or deleting the gene(s) responsible for converting acetyl-CoA to acetate and/or for converting butyryl-CoA to butyrate or by potentiating or reinforcing the genes responsible for converting acetyl-CoA to ethanoic acid or butyryl-CoA to butanoic acid. This may readily be achieved by conventional techniques, such as gene disruption, knock-out mutagenesis or negative enzyme evolution. Likewise, the microorganism may be transfected with a plasmid capable of generating anti-sense mRNA to block production of undesired enzymes, e.g. enzymes promoting ethanoic acid production when butanoic acid production is desired, and the like. It is also particularly preferred to utilize a genetically modified form of a microorganism capable of producing both ethanoic acid and butanoic acid, the genetic modification being to knock out (i.e. disable) or delete a gene responsible for the ethanoic acid or the butanoic acid production. In the case of Clostridium for example this may involve knocking out or deleting the gene(s) responsible for converting acetyl-CoA to ethanoic acid or for converting butyryl-CoA to butanoic acid or for converting acetyl-CoA to butyryl-CoA or by potentiating or reinforcing the genes responsible for converting acetyl-CoA to ethanoic acid or butyryl-CoA to butanoic acid. This again may easily be achieved by conventional techniques.

Thus, typically, supplementation of genes for the enzymes acetaldehyde dehydrogenase or ethanol dehydrogenase may lead to enhanced ethanoic acid production as may deletion, disablement or suppression of the genes for the enzymes phosphotransacetylase, acetate kinase, thiolase, acetoacetyl-CoA: acetate/butyrate coenzyme-A transferase, acetoacetate decarboxylase, 3-hydroxybutyryl-CoA dehydrogenase, crotonase, butyryl-CoA dehydrogenase, phosphotransbutyrylase, butyrate kinase, butyraldehyde dehydrogenase, aldehyde/alcohol dehydrogenase E, and butanol dehydrogenase or disablement of these enzymes or disablement of RNA coding therefore using antisense RNA. Likewise, supplementation of genes for the enzymes butyraldehyde dehydrogenase, aldehyde/alcohol dehydrogenase E, or butanol dehydrogenase, and optionally thiolase, 3-hydroxybutyryl-CoA dehydrogenase, crotonase, and butyryl-CoA dehydrogenase may lead to enhanced butanoic acid production as may deletion, disablement or suppression of the genes for the enzymes acetaldehyde dehydrogenase, ethanol dehydrogenase, phosphotransacetylase, acetate kinase, acetoacetyl-CoA: acetate/butyrate coenzyme-A transferase, acetoacetate decarboxylase, phosphotransbutyrylase, and butyrate kinase or disablement of these enzymes or disablement of RNA coding therefore using antisense RNA.

Suitable starting species for such manipulation to enhance carboxylic acid, e.g. butanoic acid, production include Clostridium thermobutyricum, C. thermopalmarium, C. thermocopriae, C. thermosaccharolyticum, Eubacterium limosum, Thermohydrogenium kirishiense, Pseudoamibacter alactolyticus, Thermobacteriodes acetoethylicus, Thermoanaerobium lactyloethylicum, Thermoproteus uzoniensis, Pyrodictium abyssi, Hyperthermus butylicus, Thermococcus stetteri and Butyribacterium methylotrophicum.

The process of converting sugars into carboxylic acid using a microorganism may be carried out in conventional conditions and can be readily determined by a skilled man. Thus the temperature will typically be in the range 20 to 45° C., more preferably 30 to 40° C., e.g. about 37° C. The pressure is preferably atmospheric pressure, although there may be an overpressure due to the presence of inert gas, e.g. N₂. The reactor used to carry out the conversion may be any conventional bioreactor, e.g. fermenter. Suitable reactors are commercially available.

The microorganism used to convert a sugar into a carboxylic acid may also be a thermophilic microorganism capable of achieving this. The use of such organisms is advantageous as it may eliminate the need to sterilise the starting material and/or yield higher conversion to acid with fewer bi products. Thermophilic microorganisms for carboxylic acid production may be identified by cultivating candidates, e.g. yeasts or Clostridium strains at the desired operating temperatures of the process, or alternately but less preferably at successively higher temperatures from a lower but elevated temperature up to the desired operating temperatures, e.g. raising the temperature in 5° C. increments from 40° C. to the desired operating temperatures. Thermophilic Clostridium strains are already known, e.g. C. thermocellum, C. fervidus, C. thermosulfurogenes, C. thermohydrosulfuricum, C. caminithermale, C. stercorarium, C. josui, C. thermolacticum, C. thermocopriae, C. straminisolvens, C. thermopapyroliticum, C. thermobutyricum, C. thermopalmarium and C. thermosaccharolyticum (see for example Mendez et al., Int. J. Syst. Bacteriol. 41:281-283 (1991), Jin et al., Int. J Syst. Bacteriol. 38:279-281 (1998), Le Ruyet et al., Syst. Appl. Microbiol. 6:196-202 (1985), Madden, Int. J. Syst. Bacteriol. 33:837-840 (1983), Hyun et al., J. Bacteriol. 156:1332-1337 (1983), Ng et al., Arch. Microbiol. 114:1-7 (1977), Wigel et al., J. Ind. Microbiol. and Biotech. 24:7-13 (2000), Lawson et al., Syst. Appl. Microbiol. 14:135-139 (1991), Hollaus et al., Arch. Micriobiol. 86:129-146 (1972) and McClung, J. Bacteriol. 29:189-202 (1935)). One such strain is deposited at the South American Biotechnology and Applied Microbiology Culture Collection as UBA 305. Other thermophilic microorganism species capable of fermenting at least some of the sugars to form useful carboxylic acids, include Thermohydrogenium kirishiense (see Zacharova et al., Arch. Microbiol. 160:492-497 (1993)), Thermobacteriodes acetoethylicus (see Ben-Basset et al., Arch. Microbiol. 128:365-370 (1981)), Thermoanaerobium lactoethylicum (see Kondratieva et al., Arch. Microbiol. 151:117-122 (1989)), Butyribacterium methylotrophicum (see Wordet at al., Fuel 70 (1990)), Thermococcus stetteri, Oxobacter pfennigii, Burkholderia xenovorans and, less preferably, Pyrodictiuim abyssi (see Pley et al., Syst. Appl. Microbiol. 14:245-253 (1991)) and Hyperthermus butylicus (see Zillig et al., J. Bacteriol. 172:3959-3965 (1990)).

Preferred thermophilic microorganisms useful in the process of the invention for generating carboxylic acids, for breaking down biomass to produce sugars for carboxylic acid generation or as starting materials for modification as described above include: Clostridium acetobutylicum (grows at 37° C.); C. beijerinckii (grows at 35° C.); C. josui (breaks down cellubiose, esculin and xylose, grows at 45° C., pH 7.0); C. thermocopriae (breaks down cellubiose and a variety of sugars, grows at 60° C., pH 6.5-7.3); C. thermosaccharolyticum (breaks down sucrose, dextrin, and pectin, grows at 55-62° C.); C. thermohydrosulfuricum (breaks down starch, cellubiose, glucose, xylose and soluble sugars, grows at 60° C., pH 6.9-7.5); C. thermobutyricum (breaks down soluble sugars, grows at 55° C., pH 6.8-7.1); C. thermopalmarium (breaks down sugars, grows at 55° C., pH 6.6); C. carboxidivorans (breaks down glucose, starch, cellulose, cellubiose and pectin, grows at 38° C., pH 6.2); Thermobacteroides acetoethylicus (breaks down starch, glucose and other soluble sugars, grows at 65° C., pH 5.5-8.5); Thermoanaerobium lactoethylicum (breaks down starch, glucose and other sugars, grows at 65° C., pH 7.0); Pyrodictium abyssi (breaks down starch and gelatin, grows at 97° C., pH 5.5); Thermococcus stetteri (breaks down peptone, starch and peptin, grows at 73-77° C., pH 6.5); Oxobacter pfennigii (grows at 36-38° C., pH 7.3); Butyribacteriuim methylotrophicum (grows at 37° C., pH 6.0); and Burkholderia xenovorans).

Further examples of thermophilic microorganisms useful in the process of the invention for generating butanoic acid, for breaking down biomass to produce sugars for butanoic acid generation or as starting materials for modification as described above include: Clostridium thermosaccharolyticum ATCC 7956 (grows at 45° C.); C. thermopalmarium DSM 5974 (grows at 55° C.); C. carboxidivorans ATCC BAA-624 (grows at up to 40° C.); Thermoanaerobacter acetoethylicus ATCC 33265 (grows at 60° C.); Thermococcus stetteri DSM 5262 (grows at 75° C.); and Oxobacter pfennigii DSM 3222 (grows at 37° C.). All of these have an acid generating phase followed by a solvent (butanol) generating phase; however the initial butyrate generation is especially effective for C. carboxidivorans, T. acetoethylicus and, especially, O. pfennigii.

In a preferred process of the invention steps (ia) and (ib) are carried out using one or more thermophilic microorganisms at a temperature of at least 45° C., more preferably at least 50° C., particularly at least 60° C., e.g. 60-80° C. In other words both carbon source to sugar conversion and sugar to carboxylic acid or a derivative thereof conversion is achieved using a thermophilic microorganism. Such processes are highly advantageous as they are isothermal. Thus energy consuming heating and cooling steps are avoided.

Particularly preferably these conversions may be carried out in a single step using a combination of thermophilic microorganisms. In this case, the different microorganisms may be added to the carbon source, e.g. biomass, simultaneously or sequentially and optionally repeatedly as desired. A single step process is advantageous as it eliminates the need to transfer sugars between reactors.

Where the microorganisms used in the hereinbefore described processes are anaerobic, the relevant process step is preferably performed under an oxygen-free or oxygen-depleted atmosphere (e.g. containing 0 to 10 mole % oxygen, preferably 0 to 5 mole %, especially 0 to 2 mole %). In this way competition for nutritional resources by aerobic microorganisms is restricted. Particularly preferably the carbon source being treated, e.g. biomass or sugars, is treated to reduce oxygen content, for example by exposure to reduced pressure or by flushing with a non-oxygen gas such as nitrogen, carbon dioxide or a noble gas.

Thus in a conversion of carbon source (e.g. biomass or sugars) to carboxylic acid or a derivative thereof, a reactor may be sterilised and sparged with an inert gas, e.g. N₂. An aqueous biomass suspension or sugar solution (e.g. from a (ia)) is asceptically introduced into the reactor and heated to an appropriate temperature depending on the microorganism being used. Optionally a culture medium is additionally added in order to faciliate growth. The skilled man will readily be able to determine suitable media. Suitable media are commercially available. A buffer may also be optionally added to control the pH of the reaction.

To start the fermentation, an aqueous inoculate of microorganism is added and the mixture agitated. If necessary, further additions of microorganism may be made, e.g. at regular intervals. Samples of the fermentation broth may be taken to assess the conversion to carboxylic acid. Typical reaction times are 12 to 72 hours, more preferably 16 to 48 hours, e.g. about 18 to 24 hours. Preferably the reactor is equipped with a heater jacket so the temperature can be controlled. Once the reaction is complete, insoluble material is preferably removed by filtration. An aqueous solution of carboxylic acid or a derivative thereof is typically obtained.

The microorganisms used in the above processes may be in non-immobilised or immobilised form. For example, the microorganisms may be immobilised on a substrate or encapsulated. The skilled man can readily produce immobilised microorganisms. The use of immobilised microorganisms is convenient as it faciliates their separation from the resulting solutions comprising sugars and/or carboxylic acid. Preferably the resulting solution is simply filtered, e.g. prior to electrochemical reduction. Thus in a preferred process, e.g. when immobilised microorganisms are used, the electrochemical reduction may be carried out on the fermentation broth produced in step (i) without purification other than filtration.

The conversion of a carbon source, e.g. biomass, to yield carboxylic acids or derivatives thereof may be carried out in a batch, semi continuous or continuous mode. Suitable reactors are commercially available.

The carboxylic acid or derivative thereof produced in step (i) is converted to an alkanol in an electrochemical reduction. As used herein the term “electrochemical reduction” refers to a process wherein a carboxylic acid or derivative thereof is reduced to its corresponding alkanol by electrolysis. It is also sometimes referred to as electroreduction. Thus an electric current is used to cause the reduction of acid to alkanol to occur. The term “electrochemical reduction” does not encompass methods wherein hydrogen is produced by electrolysis and then used to carry out a reduction.

The electrochemical reduction used in the process of the present invention may therefore be regarded as a direct electrochemical reduction.

The carboxylic acid or derivative thereof produced in step (i) is preferably transferred to an electrochemical cell. During the transfer a filtration may optionally be carried out. The concentration of carboxylic acid in the aqueous solution introduced into the electrochemical cell is typically in the range 10-1000 g/I, more preferably 20-500 g/I, e.g. 50-100 g/I.

A simplified description of the reactions that are believed to occur in the electrochemical reduction of the present invention are shown below for butanoic acid/1-butanol.

CH₃(CH₂)₂COOH+4e⁻+4H⁺→CH₃(CH₂)₂CH₂OH+H₂O Cathode reaction:

2H₂O→O₂+4e⁻+4H⁺ Anode reaction:

CH₃(CH₂)₂COOH+H₂O→CH₃(CH₂)₂CH₂OH+O₂ Overall reaction:

Any conventional electrochemical cell may be used to carry out the electrochemical reduction. A typical electrochemical cell comprises two electrodes, a cathode and an anode, an electrolyte and a power supply (i.e. current supply). The electrodes may be made of any electrically conducting material stable in the conditions, e.g. metal, graphite or semiconductor. For instance, the cathode may comprise Pt, Pd or other platinum group metals (PGM), conducting oxides of noble metals (e.g. electronically conducting mixed oxides with RuO₂) or non-noble metals, transition metals or carbon, preferably a PGM or mixed oxide where one of the components is a PGM. Similarly the anode may comprise carbon, transition metal, noble metals, noble metal oxides or oxides or mixed oxides, preferably iridium oxide based mixed oxides. Preferably the spacing between the anode and the cathode is as small as possible in order to reduce ohmic losses, but significant enough to provide sufficient separation of anode and cathode products. A Zero gap or close gap configuration is a suitable configuration. Alternatively an electrically conducting membrane with the electrodes attached to each side thereof may be advantageously be used. Preferably the cathode and anode are separated by a membrane or a diaphragm.

The electrolyte used in the electrochemical reduction of the present invention may be a liquid or solid electrolyte. Preferably the electrolyte is an aqueous solution or a proton conducting solid polymer, e.g a proton conducting polymer. In order to increase the conductivity of the cell, it may be beneficial to add a salt to the electrolyte. Suitable salts include KCl, NaCl, CaCl₂, MgCl₂ and mixtures thereof. Preferably the cell comprises means to agitate the electrolyte during the reduction process, e.g. a stirrer.

In the electrochemical reduction the current density is preferably in the range 1-200 A/dm², more preferably 2-100 A/dm², e.g. 5-50 A/dm². Typically the electrochemical reduction is carried out at a temperature in the range 25 to 100° C., more preferably 35 to 90° C., still more preferably 50 to 80° C. Preferably the electrochemical reduction is carried out at a pressure of 1 to 100 atmospheres, e.g. 2 to 20 atmospheres. Compared to the temperatures and pressures used for hydrogenation, these conditions are mild.

During the electrochemical reduction, oxygen evolves at the anode as a result of electrolysis of water. At the cathode, hydrogen generation also occurs as a side reaction in addition to the reduction of acid to alkanol. It is therefore important to avoid the mixing of oxygen and hydrogen. This is preferably achieved by using a membrane or diaphragm between the electrodes and/or by blowing an inert gas into the electrochemical cell to withdraw gases therefrom. As described below, this may also serve as a convenient way to remove alkanol from the reaction.

The use of electrochemical reduction in the process of the present invention is highly advantageous because, unlike processes utilising microorganisms, the reaction is not limited by the production of alkanol. Thus if a high concentration of carboxylic acid or a derivative thereof is introduced into the cell, a high concentration of alkanol may be achieved. The high concentration of acid drives the equilibrium of the electrochemical reaction to yield high concentrations of alkanol. In particularly preferred processes, the alkanol is removed from the electrochemical cell, therefore enabling a high conversion of acid to alkanol to be achieved.

Alkanol removal from the electrochemical cell is preferably affected by withdrawing the gas from above the electrochemical cell and condensing the alkanol from the withdrawn gas. This is especially preferred where the alkanol to be produced is methanol or, especially ethanol. Apparatus for performing such an alkanol removal preferably comprises: an electrochemical cell having a heater; a condenser; and a gas conduit from said cell to said condenser and optionally back to said cell. The apparatus is preferably provided with a pump to facilitate gas flow from the electrochemical cell to the condenser, with a cooler (e.g. a cooling jacket) for the condenser, and with an outlet port in the condenser for removal of condensed liquid therefrom.

In the above process, an inert gas, e.g. nitrogen, hydrogen or carbon dioxide, is preferably passed through the electrochemical cell so as to increase the alkanol content of the gaseous atmosphere removed from the cell. This gas may typically be the gas withdrawn from the electrochemical cell subsequent to its passage through the condenser. Gas withdrawal, moreover, moves the alkanol production reaction equilibrium so as to increase alkanol production.

Alternatively, alkanol may be removed from the electrochemical cell so as to drive the reaction to higher alkanol production by the use of selective membranes or pervapouration techniques.

In particularly preferred processes of the invention, the electrochemical reduction yields a biphasic product comprising a water phase and an alkanol (e.g. butanol) phase. The phases may form in the electrochemical cell or in a separation unit into which product from the electrochemical cell is transferred. Optionally the condensate from an apparatus as described above may also be fed into the separation unit.

Generally the water phase comprises less than 5% weight alkanol, still more preferably less than 3% weight alkanol, e.g. less than 2% weight alkanol. Correspondingly the water phase preferably comprises at least 95% weight water, more preferably at least 97% weight water, still more preferably at least 98% weight water. Generally the alkanol phase comprises at least 35% weight alkanol, still more preferably at least 40% weight alkanol, e.g. 45-50% weight alkanol. The formation of a biphasic product is highly beneficial as it facilitates isolation of the alkanol, e.g. butanol. Thus preferred processes of the invention comprise the further step of isolating said alkanol by a phase separation. In this step, the water phase is separated from the alkanol, e.g. butanol, phase by a simple phase separation. The alkanol can then be isolated from the alkanol, e.g. butanol, phase by, e.g. membrane separation (e.g. pervaporation) or distillation. Since the concentration of alkanol in this phase is much higher than that which results from the reaction, however, much less energy is required to isolate alkanol. Preferably distillation is carried out on an aqueous solution comprising 20-70% by weight alkanol, more preferably 30-50% by weight alkanol.

In the preferred processes of the present invention, steps (i) and (ii) may be carried out in the same reactor or in separate reactors. Preferably steps (i) and (ii) are carried out in separate reactors. Preferred processes of the invention comprise the further step of filtering said acid in between steps (i) and (ii). Particularly preferred processes of the invention comprise the further step of upconcentrating said acid in between steps (i) and (ii). Particularly preferably upconcentration is carried out by electrophoresis or electrodialysis, e.g. through an ion selective membrane. Both anion and cation selective membranes may be employed. Anion selective membranes are generally preferred.

Thus in preferred processes of the invention the carboxylic acid or derivative thereof produced in step (i) is upconcentrated, e.g. by electrophoresis or electrodialysis, prior to conversion to an alkanol by electrochemical reduction. Thus preferably the solution of carboxylic acid or derivative thereof produced in step (i) is transferred to an electrophoresis or electrodialysis cell comprising, e.g. an anion selective membrane and/or a cation selective membrane. The application of a direct current across the electrophoresis or electrodialysis cell causes the carboxylic acid or derivatives thereof to pass through the membrane resulting in the production of a carboxylic acid enriched solution on one side of the membrane. The enriched carboxylic acid or derivative thereof solution can be transported from the electrophoresis or electrodialysis cell to undergo electrochemical reduction as described above. The higher concentration of carboxylic acid in the enriched solution ensures a high conversion to alkanol can be achieved.

The processes of the present invention are preferably carried out in an apparatus comprising a first reactor for converting a carbon source to a carboxylic acid or a derivative thereof using a microorganism fluidly connected to a second reactor for electrochemically reducing said acid to said alkanol. Such a set up of reactors is itself novel, thus an apparatus comprising said first and second reactors, wherein said first reactor is fluidly connected to said second reactor forms a further aspect of the invention. Preferably said first reactor is a bioreactor (e.g. a fermenter). Preferably said second reactor is an electrochemical cell. The first and second reactors may be integrated into a single unit. More preferably the first and second reactors are separate units.

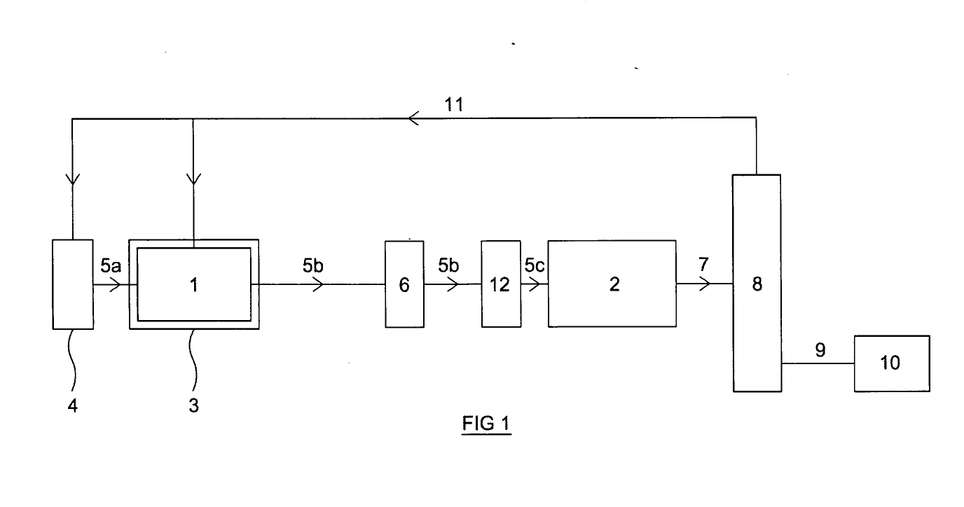

FIG. 1 is a schematic diagram of a preferred apparatus according to the present invention. Referring to FIG. 1, a first reactor 1 wherein carboxylic acid is produced is fluidly connected to a second reactor 2 wherein said acid is reduced to alkanol. First reactor 1 is preferably a fermentation vessel equipped with a heating jacket 3. First reactor 1 may optionally be fluidly connected to pretreatment vessel 4 by conduit 5a wherein biomass may be converted to sugars, e.g. fermentable sugars. Conduit 5b leads from first reactor 1, via an optional filtration unit 6 and an optional upconcentration unit 12, to second reactor 2. Second reactor 2 is preferably an electrochemical cell. Conduit 7 leads from second reactor 2 to a separation unit 8 wherein phase separation may occur. Conduit 9 leads to collection vessel 10 from which an enriched alkanol, e.g. butanol, phase may be collected. Return conduit 11 leads back to the reactor 1. The transfer of liquids between reactors and units is preferably achieved using pumps (not shown).

Particularly preferred apparatus for carrying out the processes of the present invention comprise a unit 12 for upconcentrating said carboxylic acid or derivative thereof. Referring to FIG. 1, the unit 12 when present is located in between filtration unit 6 and second reactor 2. Preferably the unit 12 is an electrophoretic cell.

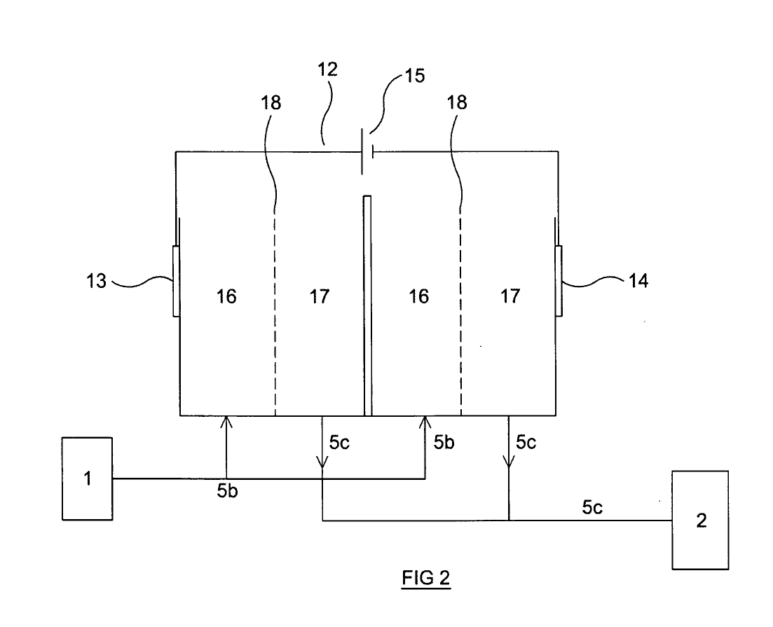

FIG. 2 is a schematic of a preferred electrophoretic cell 12. Referring to FIG. 2, the electrophoretic cell 12 comprises anode 13, cathode 14 and current supply 15 that are used to apply a direct current across the cell. Preferably the anode and cathode do not contact the solution. The cell 12 comprises at least one first compartment 16 and at least one second compartment 17. Each of first 16 and second 17 compartments are separated by an anion selective membrane 18. A solution of carboxylic acid or derivative thereof is introduced into the cell 12 via conduit 5a, 5b. When direct current from supply 15 is applied the carboxylate anions present in the solution produced from fermentation are drawn through the membrane into said second compartments 17. Thus the affect of applying direct current to the cell 12 is to produce carboxylic acid enriched solution in second compartments 17. This enriched solution may be transferred via conduit 5c to second reactor 2. As shown in FIG. 2, an electrophoretic cell may comprise more than one first compartment, second compartment and selective membrane. In this case, enriched solution is transferred from each second compartment via conduit 5c to the second reactor 2. No chemical transformation occurs during the separation process.

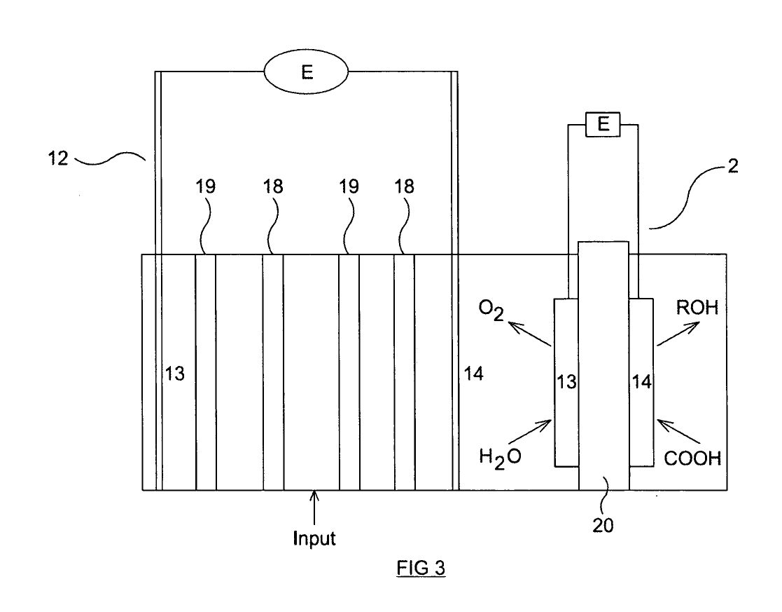

FIG. 3 is a schematic of another preferred electrophoretic cell for carrying out the processes of the invention. Referring to FIG. 3, the electrophoretic cell 12 and second reactor 2 is separated by anion selective membranes 18. Cell 12 also comprises cation selective membranes 19. Conversion of a carbon source to carboxylic acid is carried out in a reactor 1 (not shown), then the solution is transferred to electrophoretic cell 12. A current applied across the cell via electrodes 13 and 14 causes the carboxylate ions present in the solution to pass through membranes 18 into the second reactor 2. Electrochemical reduction is carried out in second reactor 2. Second reactor 2 shown in FIG. 3 comprises a diaphragm 20 to which electrodes 13, 14 are attached. The diaphragm separates the products produced in the electroreduction reaction.

1. A process for the production of an alkanol comprising:
(i) converting a carbon source into a carboxylic acid or a derivative thereof using a microorganism; and
(ii) electrochemically reducing said acid to said alkanol.

2. A process as claimed in claim 1, wherein said acid is butanoic acid and said alkanol is butanol.

3. A process as claimed in claim 1, wherein said carbon source is biomass, preferably plant biomass.

4. A process as claimed in claim 1, wherein step (i) comprises the steps of:
(ia) converting a carbon source into sugars; and
(ib) treating said sugars with said microorganism to yield said carboxylic acid or a derivative thereof

5. A process as claimed in claim 4, wherein said said carbon source is converted into sugars by a process comprising an acid hydrolysis treatment, a hydrothermal treatment, a chemical treatment, an enzymatic hydrolysis or any combination of the afore-going.

6. A process as claimed in claim 4, wherein said carbon source is converted into sugars by a process comprising treating said carbon source with a thermophilic microorganism at a temperature of at least 45° C.

7. A process as claimed in claim 6, wherein said thermophilic microorganism is a Clostridium species.

8. A process as claimed in claim 1, wherein said carbon source, preferably sugars, is converted into said carboxylic acid or derivative thereof using a Clostridium species, e.g. C. acetobutylicum, C. aurantibutyricum, C. beijerinckii, C. butyicum, C. kluyveri, C. pastuerianum, C. saccharobutylicum, C. saccharolyticum, C. thermobutyricum, C. thermocellum, C. thermosaccarolyticum and C. tyrobutyricum.

9. A process as claimed in claim 8, wherein said Clostridium species is

10. A process as claimed in claim 4, wherein steps (ia) and (ib) are carried out using one or more thermophilic microorganisms at a temperature of at least 45° C.

11. A process as claimed in claim 1, wherein said electrochemical reduction yields a biphasic product comprising a water phase and an alkanol (e.g. butanol) phase.

12. A process as claimed in claim 1, comprising a further step of isolating said alkanol using a phase separation.

13. A process as claimed in claim 1, comprising a further step of isolating said alkanol by distillation.

14. A process as claimed in claim 1, wherein steps (i) and (ii) are carried out in separate reactors.

15. A process as claimed in claim 1, comprising the further step of upconcentrating said carboxylic acid in between steps (i) and (ii).

16. A process as claimed in claim 15, wherein said upconcentration is carried out by electrophoresis.

17. An apparatus for carrying out a process as defined in claim 1.

18. An apparatus for producing an alkanol comprising:
(ia) a first reactor (e.g. a fermenter) for converting a carbon source into a carboxylic acid or a derivative thereof using a microorganism; and
(ib) a second reactor (e.g. an electrochemical cell) for electrochemically reducing said acid to said alkanol,
wherein said first reactor is fluidly connected to said second reactor.

19. An apparatus as claimed in claim 18, wherein said microorganism is immobilised within said first reactor.

20. An apparatus as claimed in claim 18, wherein the electrochemical cell comprises a liquid or solid electrolyte.

21. An apparatus as claimed in claim 18, further comprising a filtration unit in between said first and second reactors.

22. An apparatus as claimed in claim 18, further comprising a unit for upconcentrating said carboxylic acid or derivative thereof.

23. An apparatus as claimed in claim 22, wherein said unit is in between said first and second reactors and is fluidly connected to both said reactors.

24. An apparatus as claimed in claim 22, wherein said unit comprises an electrophoretic or electrodialysis cell comprising at least one electrophoretic membrane (e.g. an anion selective membrane).
